(19) United States
(12) Patent Application Publication
(10) Pub. No.: US 2023/0207139 A1
(43) Pub. Date: Jun. 29, 2023

(54) A METHOD SYSTEM AND STORAGE MEDIUM FOR LOCATING SOURCE OF INFECTION BASED ON BIG DATA

(71) Applicant: Yunan Han, Beijing (CN)

(72) Inventors: Yunan Han, Beijing (CN); Qixian He, Beijing (CN)

(21) Appl. No.: 17/296,141

(22) PCT Filed: May 13, 2020
(86) PCT No.: PCT/CN2020/089912

§ 371 Date: May 21, 2021

(51) Int. Cl. G16H 50/80

(52) U.S. Cl. CPC G16H 50/80

(57) ABSTRACT

A method, a system and a storage medium for locating a source of infection based on big data. The method may include: Calculate and update person safety levels of each target person at each moment according to a plurality of parameters such as person safety levels at the previous time at the first current time, the person safety level determined based on the detection information, and the infection transmission probability; Calculate the regional risk level of the target location at the second current moment according to a plurality of parameters such as the regional risk level of the target location at the previous moment of the second current moment, the disinfection coefficient, and person safety levels, and calculate and update the regional risk level of each target location at each moment; Give an alarm to the smart mobile terminal based on a preset risk level threshold.

### TECHNICAL FIELD

The present invention relates to the technical field of big data positioning, in particular to a method, system and storage medium for locating a source of infection based on big data.

### BACKGROUND

With the development of technology, the means of obtaining information are gradually increasing. And obtaining information rapidly is very important for the prevention and control of the epidemic. In existing methods, the epidemic prevention and control measures are mainly to restrict interpersonal communication, which can restrain the spread of epidemic to a certain extent. But it will also have a negative impact on normal production and daily life. Moreover, due to the limited information, the above measures cannot be specific to every person, every place and every moment.

In the existing epidemic prevention and control measures, the epidemic map can be viewed through the epidemic prevention and control software installed in the smart mobile terminal including the community information where the epidemic occurs. However, it unable to track the movement of infection spreading crowd nor does it have real-time warning functions. In addition, you can also send the health code via SMS to the operator, or obtain the health code via WeChat, etc., so that you can obtain which provinces and cities the smart mobile terminal has visited in the past period of time. However, the data is inaccurate and cannot specifically reflect contact with the source of infection

### SUMMARY

In view of this, a method, system, and storage medium for locating the source of infection based on big data are provided to solve the situation that the source of infection cannot be accurately located and the epidemic prevention and control measures are not in place.

The present invention adopts the following technical solutions:

In the first aspect, an embodiment of the present application provides a method for locating a source of infection based on big data, which includes following parts:

Calculate and update the person safety level of each target person at each moment according to the person safety level at the previous time at the first current time, the person safety level determined based on the detection information, the infection transmission probability at the previous time at the first current time, the regional risk level of the previous moment of the first current moment and the infection probability of the environment where the target person is exposed to; Wherein, the first current moment and the previous moment of the first current moment are separated by a first time period, and the initial person safety level of each target person is preset;

Calculate the regional risk level of the target location at the second current moment according to the regional risk level of the target location at the previous moment of the second current moment, the disinfection coefficient at the previous time of the second current time, the person safety level at the previous moment of the second current moment, the transmission probability of the infected person to the environment and the infection source dissipation coefficient. So as to calculate and update the regional risk level of each target location at each moment; Wherein, the second current moment and the previous moment of the second current moment are separated by a second time period, and the initial regional risk level of each target location is preset;

Give an alarm to the smart mobile terminal with a regional risk level greater than the set regional risk level threshold, and/or, give an alarm when a smart mobile terminal with a person safety level less than the set person safety level threshold enters the set Bluetooth interconnection range.

In the second aspect, an embodiment of the present application provides a system for locating a source of infection, which includes a server and at least one smart mobile terminal:

Wherein the server is used to obtain and update a map library, wherein the map library stores the regional risk levels of each location at each time;

The server is used to obtain and update a person database, wherein the person database stores the person safety level of each person at each time;

The smart mobile terminal is used to obtain basic person information and person safety level, and update the person safety level;

The smart mobile terminal is used to display a map interface in real time, wherein the application uses set various colors to mark the regional risk level of each location;

The smart mobile terminal is used to query the historical track of person and person contact history information;

The smart mobile terminal is used to display the person safety level obtained within the set distance of each smart mobile terminal.

In a third aspect, an embodiment of the present application provides a storage medium that stores a computer program. And when the computer program is executed by a processor, the steps in the method for locating the source of infection based on big data as described in the first aspect are realized.

The present invention adopts the above technical solutions and does not rely strongly on infectious disease monitoring. When there is no infectious disease monitoring, the method can still calculate and update the regional risk level of each location at each time and the safety level of each person at each moment based on the big data of the flow of people. And the alarm function is provided when entering a high-risk area or when a high-risk person enters the Bluetooth interconnection range.

### BRIEF DESCRIPTION OF THE DRAWINGS

FIG. 1 is a flow chart of the method for locating the source of infection based on big data according to an embodiment of the present application;

FIG. 2 is a schematic diagram of the structure of a big data-based infection source location system according to an embodiment of the present application;

FIG. 3 is a schematic structural diagram of a big data-based infection source location system applicable in an embodiment of the present application;

FIG. 4 is a schematic diagram of a Bluetooth positioning algorithm applicable in an embodiment of the present application.

### DESCRIPTION OF THE EMBODIMENTS

In order to make the purpose, technical solutions and advantages of the present invention clearer, the technical solutions of the present invention will be will be further described in detail below. Obviously, the described embodiments are only a part of the embodiments of the present invention, rather than all the embodiments. Based on the embodiments of the present invention, all other implementations obtained by those of ordinary skill in the art without creative work shall fall within the protection scope of the present invention.

First, make an explain to the applicable scenarios of the embodiments of the present application. The method of locating the source of infection can be applied to the control of infectious diseases. The method can also be used to intelligently generate epidemic investigation reports to guide epidemic prevention and control.

### EMBODIMENTS

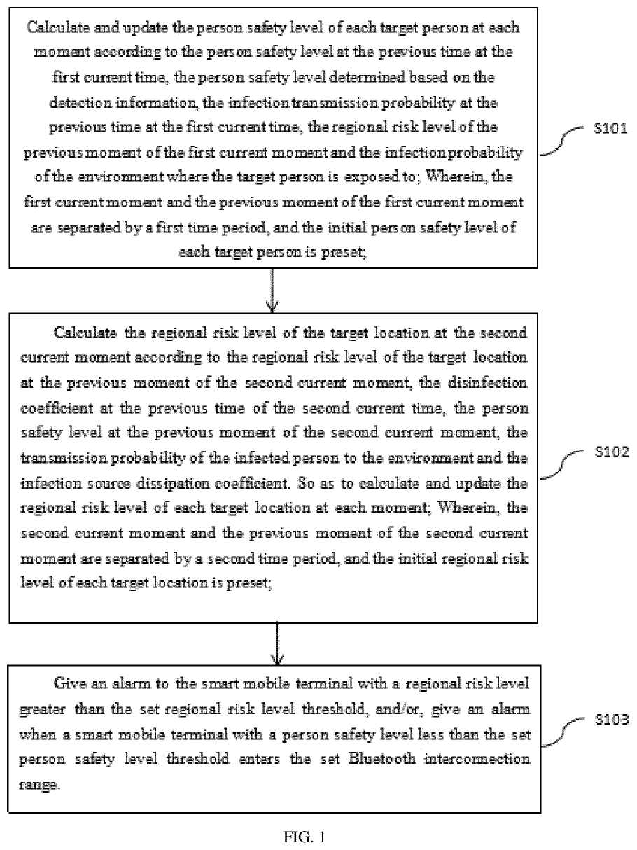

FIG. 1 is a flowchart of a method for locating a source of infection based on big data according to an embodiment of the present invention. The method can be executed by the system for locating a source of infection based on big data provided by the embodiment of the present invention. Referring to FIG. 1, the method may specifically include the following parts:

Structure 1

According to the person safety level at the previous time at the first current time, the person safety level determined based on the detection information, the infection transmission probability at the previous time at the first current time, the regional risk level of the previous moment of the first current moment and the infection probability of the environment where the target person is exposed to, calculate and update the person safety level of each target person at each moment; Wherein, the first current moment and the previous moment of the first current moment are separated by a first time period, and the initial person safety level of each target person is preset.

Specifically, the person safety level needs to be updated in real time, and the reasons for the update include the detection situation, the contact person's situation, and the characteristics of the contact area. Optionally, the first current moment is recorded as t, the first time period is recorded as to, and the previous moment of the first current moment is recorded as t−t₀. The initial person safety level of each target person can be set according to the initial person information of the target person, such as the initial position and initial protective measures.

In specific implementation, the calculation and update methods of the person safety level of each target person at each moment may be given by

P(t)=P(t−t₀)+D(t)+Σᵢ₌₀ⁿPᵢ(t−t₀)αᵢ(t−t₀)+S(Ø,θ,t−t₀)β, (1)

where P(t−t₀) is the safety level of the target person at the t−t₀ moment, D(t) is the safety level of person after the detection based on detection information at t moment, Pᵢ(t−t₀) is the safety level of each person obtained by the target person's smart phone through Bluetooth interconnection within the set distance, i is person number, the total number of person other than target person is n. αᵢ(t−t₀) is transmission probability for infection. S(Ø, θ, t−t₀) is the region risk level of the target person at location (Ø, θ) at time t−t₀. β is the probability of the target person being infected by the environment.

Optionally, the infection transmission probability is determined according to the relative positions of the infected person's smart mobile terminal and each smart mobile terminal, and the protective measures of the infected person.

Optionally, the probability of the target person being infected by the environment is determined based on the target person's protective measures and the target person's stay time in the environment.

Exemplarily, the calculation method of the distance between two smart mobile terminals may be given by

$$Dist ( {RSSI}_{1} , {RSSI}_{2} ) = {10}^{\frac{\vert {RSSI}_{1} + {RSSI}_{2} \vert / 2 - {A}_{1}}{10 {A}_{tt}}} + δ , \qquad ( 2 )$$

where Dist is the distance between two devices, RSSI₁ is the signal strength from the Bluetooth port of the second mobile terminal to the first one, RSSI₂ is the signal strength from the Bluetooth port of the first mobile terminal to the second one, A₁ is the Bluetooth channel attenuation coefficient under the standard 1 meter distance, Aₜₜ is environmental attenuation factor, S is environmental correction parameters.

Specifically, the Bluetooth RSSI (Received Signal Strength Indication, received signal strength indication) is converted into distance by fitting regression function, which is similar to the change law of RSSI. In a specific example, the above distance Dist is the estimated distance between two smart mobile terminals. Under normal circumstances, according to the surrounding environment, the value of A₁ can be given as 59, the value of Aₜₜ is 2, and the value δ is 0.2.

In specific implementation, the method for obtaining location information when the human or animal is the carrier of the source of infection in this embodiment is as follows:

Through GPS (Global Positioning System, Global Positioning System) positioning, the position of the smart mobile terminal can be obtained. And then, the beacon node can be positioned by GPS. Furthermore, a more accurate relative position can be obtained by Bluetooth positioning.

In specific implementation, FIG. 4 shows a schematic diagram of a Bluetooth positioning algorithm; as shown in FIG. 4, there are three non-collinear beacon nodes A, B, C with known coordinates and one unknown node D. Among them, A, B, and C are all within the communication radius of Dist. The coordinates of the three beacon nodes are (x₁, y₁), (x₂, y₂), (x₃, y₃), and then from equation (2), the distances from the three beacon nodes to the unknown node can be calculated as d₁, d₂, d₃. Let the coordinates of the unknown node D be (x, y), so,

(x−x₁)²+(y−y₁)²=d₁², (x−x₂)²+(y−y₂)²=d₂², (x−x₃)²+(y−y₃)²=d₃² (3)

Take the difference of the square of the distance, and subtract the equations in equation (3) respectively, and then the equations of three straight lines, l₁, l₂ and l₃ in FIG. 4 can be obtained:

2(x₂−x₁)x+2(y₂−y₁)y=d₁²−d₂²−x₁²+x₂²−y₁²+y₂², 2(x₃−x₂)x+2(y₃−y₂)y=d₂²−d₃²−x₂²+x₃²−y₂²+y₃², 2(x₃−x₁)x+2(y₃−y₁)y=d₁²−d₃²−x₁²+y₃² 4

After processing the above equation (4), the unknown node coordinates (x, y) can be written as

$$[ \begin{matrix}x \\ y\end{matrix} ] = {[ \begin{matrix}2 ( 2 {x}_{2} - {x}_{1} - {x}_{3} ) 2 ( 2 {y}_{2} - {y}_{1} - {y}_{3} ) \\ 2 ( 2 {x}_{3} - {x}_{2} - {x}_{1} ) 2 ( 2 {y}_{3} - {y}_{1} - {y}_{2} )\end{matrix} ]}^{- 1} [ \begin{matrix}{d}_{1}^{2} - 2 {d}_{2}^{2} + {d}_{3}^{2} - {x}_{1}^{2} + 2 {x}_{2}^{2} - {x}_{3}^{2} - {y}_{1}^{2} + 2 {y}_{2}^{2} - {y}_{3}^{2} \\ {d}_{1}^{2} + {d}_{2}^{2} + 2 {d}_{3}^{2} - {x}_{1}^{2} - {x}_{2}^{2} + 2 {x}_{3}^{2} - {y}_{1}^{2} - {y}_{2}^{2} + 2 {y}_{3}^{2}\end{matrix} ] . \qquad ( 5 )$$

By combining the three beacon nodes into one group, the possible location of an unknown node can be obtained. If the beacon nodes meeting the conditions around the unknown node are divided into a group for every three, the possible positions of m unknown nodes can be obtained.

In order to obtain the position of the unknown node more accurately, use the weighting coefficient method to determine the weight of the coordinates involved in the calculation. Suppose that

f(x,y)=Σᵢ₌₁^{m}(x−xᵢ)²+(y−yᵢ)², (6)

among them,

m=Cₘ³. (7)

The partial derivatives of x and y in equation (6) can be obtained as

$$\frac{\partial f ( x , y )}{\partial x} = m x - {\sum }_{i = 1}^{m} {x}_{i} = 0 , \qquad ( 8 )$$

$$\frac{\partial f ( x , y )}{\partial y} = m y - {\sum }_{i = 1}^{m} {y}_{i} = 0.$$

Solve the above formula to get

$$\dot{x} = \frac{1}{m} {Σ}_{i = 1}^{m} {x}_{i} , \qquad ( 9 )$$

$$\dot{y} = \frac{1}{m} {Σ}_{i = 1}^{m} {y}_{i} .$$

Here, point P(\dot{x}, \dot{y}) is an unbiased estimate of unknown node X(x, y). Use the difference between each possible location point and P point to measure the weight of each possible location point, suppose that

Δd=\sqrt{(xᵢ−\dot{x})²+(yᵢ−\dot{y})²}. (10)

So the weight coefficient of each possible location point is

$${ω}_{i} = \frac{\frac{1}{\sqrt{{( {x}_{i} - \dot{x} )}^{2} + {( {y}_{i} - \dot{y} )}^{2}}}}{{Σ}_{i = 1}^{m} \frac{1}{\sqrt{{( {x}_{i} - \dot{x} )}^{2} + {( {y}_{i} - \dot{y} )}^{2}}}} . \qquad ( 11 )$$

Finally, the final position of the unknown node is

x=Σᵢ₌₁^{m}ωᵢxᵢ, y=Σᵢ₌₁^{m}ωᵢxᵢ. (12)

The above is the method for obtaining location information when the human or animal is the carrier of the infection source, and the following is the method that location is the source of infection.

Optionally, the infection transmission probability is inversely related to the relative positions of the infected person's smart mobile terminal and each smart mobile terminal, and the infection transmission probability is related to the protective measures of the infected person.

Structure 2

According to the regional risk level of the target location at the previous moment of the second current moment, the disinfection coefficient at the previous time of the second current time, the person safety level at the previous moment of the second current moment, the transmission probability of the infected person to the environment and the infection source dissipation coefficient, calculate the regional risk level of the target location at the second current moment. So as to calculate and update the regional risk level of each target location at each moment; Wherein, the second current moment and the previous moment of the second current moment are separated by a second time period, and the initial regional risk level of each target location is preset;

Specifically, the regional risk level is updated in real time, and the reasons for the update include person contamination, disinfection measures, and the extinction of viruses over time. The second current moment is recorded as t₁, the first time period is recorded as Δt, and the previous moment of the first current moment is t₁−Δt. The initial regional risk level of each target location can be pre-set according to the relevant data provided by the disease control department.

In specific implementation, the method of calculating the regional risk level of each target location at each moment may be given by

S(Ø,θ,t₁)=C(t₁)S(Ø,θ,t₁−Δt)+e^{-τ(t}^{₂}^{t}^{₁}⁾Σᵢ₌₀ⁿ⁺¹Pᵢ(t₁−Δt)γᵢ(t₁−Δt). (13)

Here, S(Ø, θ, t₁−Δt) is the regional risk level of location (Ø, θ) at time t₁−Δt, C(t₁) is the disinfection coefficient of the virus that the virus in the current area before time t₁ is eliminated in a set ratio after disinfection measures are taken at time t₁, Pᵢ(t₁−Δt) is the person safety level of person i at time t₁−Δt, γᵢ(t₁−Δt) is the transmission probability of a infected person to the environment at time t₁−Δt, e^{-τ(t}^{₂}^{t}^{₁}⁾is the dissipation factor of the source of infection, τ is the attenuation coefficient, and t₂−t₁ is the residence time of the source of infection. The attenuation coefficient τ is related to the environment and surrounding materials.

Optionally, the transmission probability of the infected person to the environment is related to the protection measures and the safety level of the infected person.

Structure 3

Give an alarm to the smart mobile terminal with a regional risk level greater than the set regional risk level threshold, and/or, give an alarm when a smart mobile terminal with a person safety level less than the set person safety level threshold enters the set Bluetooth interconnection range.

Among them, the set Bluetooth interconnection range can be 30 meters. Specifically, in practical use, areas with a regional risk level higher than the regional risk level threshold are set as high-risk areas, and an alarm is given when the smart terminal enters the high-risk area. In addition, people whose person safety level is less than the set person safety level threshold are set as high-risk person. When high-risk person enters the set Bluetooth interconnection range, each smart mobile terminal will be alerted.

In the embodiments of this application, the method for locating the source of infection based on smart mobile terminals and cloud platforms does not strongly rely on infectious disease monitoring. When there is no infectious disease monitoring, the method can still calculate and update the regional risk level of each location at each time and the safety level of each person at each moment based on the big data of the flow of people. And the alarm function is provided when entering a high-risk area or when a high-risk person enters the Bluetooth interconnection range.

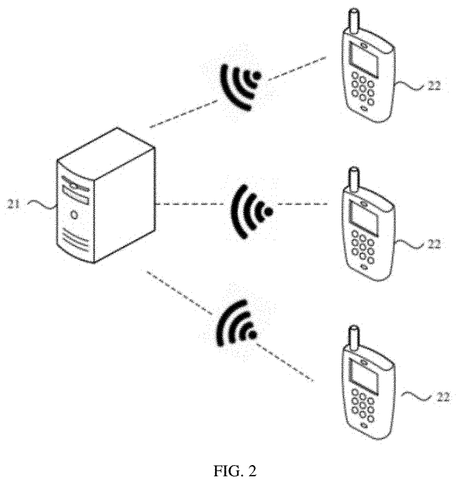

FIG. 2 is a schematic structural diagram of a system for locating a source of infection provided by an embodiment of the present invention. The system is suitable for executing a method for locating a source of infection based on big data provided by an embodiment of the present invention. As shown in FIG. 2, the system may specifically include a server 21 and at least one smart mobile terminal 22.

Wherein, the server 21 is used to obtain and update a map library, where the map library stores the regional risk levels of each location at each time; The server 21 is used to obtain and update the person database, wherein the person database stores the person safety level of each person at each moment; The smart mobile terminal 22 is used to obtain basic person information and person safety level, and to update the person safety level; The smart mobile terminal 22 is used to display the map interface in real time, wherein the map interface uses a set color to mark the regional risk level of each location; The smart mobile terminal 22 is used to query the historical movement of person and person contact history information; The smart mobile terminal 22 is used to display the person safety level obtained within the set distance of each smart mobile terminal.

Specifically, person carry one or more smart mobile terminals, which can be smart phones, smart watches, smart bracelets, etc. The smart mobile terminal installs the smart mobile terminal application software to call the Bluetooth function, mobile data function and combined positioning function of the smart terminal. It exchanges information with server application software through mobile data function, and exchanges information with smart mobile terminals carried by people around by calling the Bluetooth function. The server runs server application software, which is used to exchange information with a large number of smart mobile terminals via the Internet.

Exemplarily, the server application software can run on a cloud platform, and the map library and person database can be refreshed in real time. Each smart mobile terminal can obtain the basic information of the person, update the person safety level in real time, display the map interface in real time, query the historical track of person and the person safety level at that time, and query the person contact history information. The real-time map interface displays a real-time map and uses colors to mark the safety levels of different locations. At the same time, it displays the safety levels of surrounding people obtained by smart mobile terminals within about 30 meters through Bluetooth interconnection.

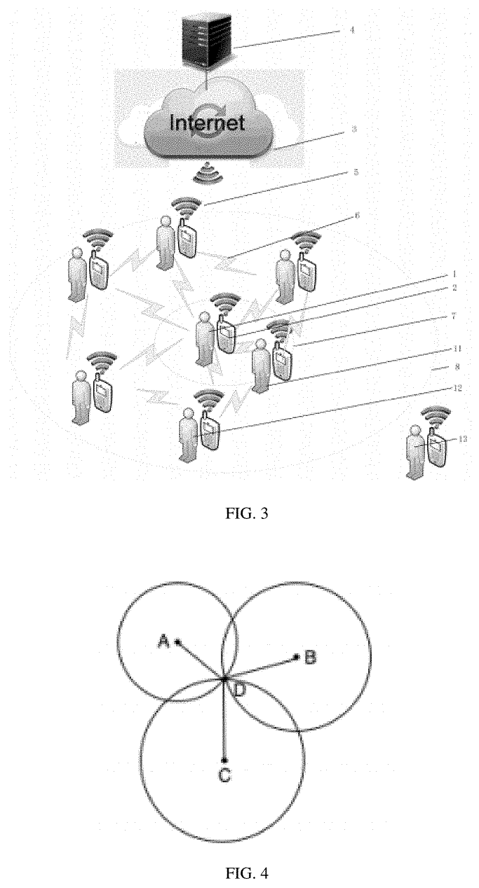

In specific implementation, FIG. 3 shows a schematic structural diagram of a big data-based infection source location system. Among them, 1 represents the target person, 2 represents the smart mobile terminal, 3 represents the Internet, 4 represents the cloud platform, 5 represents the data transmission of the mobile network, 7 represents the short distance circle of Bluetooth transmission, 8 represents the critical transmission distance circle of Bluetooth transmission, 11 indicates the people in the short-range circle of the Bluetooth transmission of the target person 1, 12 indicates the person located in the critical transmission distance circle of the target person's Bluetooth transmission, and 13 indicates the person located outside the critical transmission distance circle of the target person's Bluetooth transmission.

In the embodiments of this application, the method for locating the source of infection based on smart mobile terminals and cloud platforms does not strongly rely on infectious disease monitoring. When there is no infectious disease monitoring, the method can still calculate and update the regional risk level of each location at each time and the safety level of each person at each moment based on the big data of the flow of people. And the alarm function is provided when entering a high-risk area or when a high-risk person enters the Bluetooth interconnection range. And give the infectious disease risk distribution map of each location; According to the big data formed by the person's recent person contact, it may analyze the person's infectious disease infection risk.

Exemplarily, data information transmitted through Bluetooth communication between smart mobile terminals carried by people includes person identity information, person safety level, and Bluetooth signal strength. The smart mobile terminal application software carried by the person communicates with the cloud platform server software through mobile data, and the communication uses the mobile Internet to exchange data. The cloud platform server software transmits key information to the smart mobile terminal application software, including real-time map information, location information of high-risk person within a given distance and high-risk alarm information around the person. The smart mobile terminal application software transmits key information to the cloud platform server software, including person identity information, person safety level, person real-time location information, and the number of surrounding people and the person safety level corresponding to surrounding people obtained through the Bluetooth of the person's smart mobile terminal.

Therefore, in the embodiment of the present application, the person safety level information has a real-time update function, and the reasons for the update include the detection situation, the contact person's situation, and the characteristics of the contact area. The risk level of a region has a real-time update function. The reasons for the update include person pollution, disinfection measures, and virus apoptosis over time. The cloud platform server software records person safety information and person contact information, and can generate close contact reports of sensitive person. The smart mobile terminal application software can display the epidemic safety map in real time and display the safety level of the surrounding people. On the basis of the system, the software operator of the cloud platform server, after obtaining the authorization, can make a comprehensive assessment of the epidemic situation, accurately locate the epidemic information, and provide rich big data support for the prevention and control of the epidemic.

The system for locating the source of infection based on big data provided by the embodiment of the present invention can execute the method for locating the source of infection based on big data provided by any embodiment of the present invention, and has corresponding functional modules and beneficial effects for the execution method.

The embodiment of the present invention also provides a storage medium that stores a computer program, and when the computer program is executed by a processor, each step in the method for locating the source of infection based on big data in the embodiment of the present invention is implemented:

Calculate and update the person safety level of each target person at each moment according to the person safety level at the previous time at the first current time, the person safety level determined based on the detection information, the infection transmission probability at the previous time at the first current time, the regional risk level of the previous moment of the first current moment and the infection probability of the environment where the target person is exposed to; Wherein, the first current moment and the previous moment of the first current moment are separated by a first time period, and the initial person safety level of each target person is preset;

Calculate the regional risk level of the target location at the second current moment according to the regional risk level of the target location at the previous moment of the second current moment, the disinfection coefficient at the previous time of the second current time, the person safety level at the previous moment of the second current moment, the transmission probability of the infected person to the environment and the infection source dissipation coefficient. So as to calculate and update the regional risk level of each target location at each moment; Wherein, the second current moment and the previous moment of the second current moment are separated by a second time period, and the initial regional risk level of each target location is preset;

Give an alarm to the smart mobile terminal with a regional risk level greater than the set regional risk level threshold, and/or, give an alarm when a smart mobile terminal with a person safety level less than the set person safety level threshold enters the set Bluetooth interconnection range.

It can be understood that the same or similar parts in the foregoing embodiments may be referred to each other, and the contents not described in detail in some embodiments may refer to the same or similar contents in other embodiments.

It should be noted that in the description of the present invention, the terms “first”, “second”, etc. are only used for descriptive purposes, and cannot be understood as indicating or implying relative importance. In addition, in the description of the present invention, unless otherwise specified, the meaning of “plurality” means at least two.

Any process or method description in the flowchart or described in other ways herein can be understood as a module, segment or part of code that includes one or more executable instructions for implementing specific logical functions or steps of the process. And the scope of the preferred embodiment of the present invention includes additional implementations, which may not be in the order shown or discussed, including the execution of functions in a substantially simultaneous manner or in reverse order according to the functions involved. This should be understood by those skilled in the art to which the embodiments of the present invention belong.

It should be understood that each part of the present invention can be implemented by hardware, software, firmware or a combination thereof. In the foregoing embodiments, multiple steps or methods can be implemented by software or firmware stored in a memory and executed by a suitable instruction execution system. For example, if it is implemented by hardware, as in another embodiment, it can be implemented by any one or a combination of the following technologies known in the art: Discrete logic circuits with logic gate circuits used to implement logic functions for data signals, application-specific integrated circuits with suitable combinational logic gate circuits, programmable gate array (PGA), field programmable gate array (FPGA), etc.

Those of ordinary skill in the art can understand that all or part of the steps carried in the method of the foregoing embodiments can be implemented by a program instructing relevant hardware to complete. The program may be stored in a computer-readable storage medium, and when the program is executed, it includes one of the steps of the method embodiment or a combination thereof.

In addition, the functional units in the various embodiments of the present invention may be integrated into one processing module, or each unit may exist alone physically, or two or more units may be integrated into one module. The above-mentioned integrated modules can be implemented in the form of hardware or software functional modules. If the integrated module is implemented in the form of a software function module and sold or used as an independent product, it can also be stored in a computer readable storage medium.

The storage medium mentioned above may be a read-only memory, a magnetic disk or an optical disk, etc.

In the description of this specification, descriptions referring to the terms “one embodiment”, “some embodiments”, “examples”, “specific examples”, “some examples” or the like means that the specific features, structures, materials, or characteristics described in connection with this embodiments or examples are included in at least one embodiment or example of the present invention. In this specification, the schematic representations of the above terms do not necessarily refer to the same embodiments or examples. Furthermore, the described specific features, structures, materials or characteristics may be combined in any one or more embodiments or examples in a suitable manner.

Although the embodiments of the present invention have been shown and described above, it can be understood that the above embodiments are exemplary and cannot be construed as limitations of the present invention, and those skill in the art can make changes, modifications, substitutions and variations to the above embodiments within the scope of the present invention.

1. A method for locating the source of infection based on big data, the method comprising:
calculating and updating the person safety level of each target person at each moment according to the person safety level at the previous time at the first current time, the person safety level determined based on the detection information, the infection transmission probability at the previous time at the first current time, the regional risk level of the previous moment of the first current moment and the infection probability of the environment where the target person is exposed to; Wherein, the first current moment and the previous moment of the first current moment are separated by a first time period, and the initial person safety level of each target person is preset;
calculating the regional risk level of the target location at the second current moment according to the regional risk level of the target location at the previous moment of the second current moment, the disinfection coefficient at the previous time of the second current time, the person safety level at the previous moment of the second current moment, the transmission probability of the infected person to the environment and the infection source dissipation coefficient. So as to calculate and update the regional risk level of each target location at each moment;
Wherein, the second current moment and the previous moment of the second current moment are separated by a second time period, and the initial regional risk level of each target location is preset; and
giving an alarm to the smart mobile terminal with a regional risk level greater than the set regional risk level threshold, and/or, give an alarm when a smart mobile terminal with a person safety level less than the set person safety level threshold enters the set Bluetooth interconnection range.

2. The method of claim 1, wherein the infection transmission probability is determined according to the relative positions of the infected person's smart mobile terminal and each smart mobile terminal, and the protective measures of the infected person.

3. The method of claim 1, wherein the infection probability of the target person in the environment is determined according to the protective measures of the target person and the residence time of the target person in the environment.

4. The method of claim 1, wherein the transmission probability of the infected person to the environment is related to the protective measures of the infected person and the safety level of the infected person.

5. The method of claim 1, wherein the method for calculating and updating the person safety level of each target person at each moment may be given by
P(t)=P(t−t₀)+D(t)+Σᵢ₌₀ⁿPᵢ(t−t₀)αᵢ(t−t₀)+S(Ø,θ,t−t₀)β,
where P(t−t₀) is the safety level of the target person at the t−t₀ moment, D(t) is the safety level of person after the detection based on detection information at t moment, Pᵢ(t−t₀) is the safety level of each person obtained by the target person's smart phone through Bluetooth interconnection within the set distance, i is person number, the total number of person other than target person is n. αᵢ(t−t₀) is transmission probability for infection. S(Ø, θ, t−t₀) is the region risk level of the target person at location (Ø, θ) at time t−t₀. β is the probability of the target person being infected by the environment.

6. The method of claim 1, wherein the calculation method of the regional risk level of each target location at each time may be given by
S(Ø,θ,t₁)=C(t₁)S(Ø,θ,t₁−Δt)+e^{-τ(t}^{₂}^{t}^{₁}⁾Σᵢ₌₀ⁿ⁺¹Pᵢ(t₁−Δt)γᵢ(t₁−Δt).
Here, S(Ø, θ, t₁−Δt) is the regional risk level of location (Ø, θ) at time t₁−Δt, C(t₁) is the disinfection coefficient of the virus that the virus in the current area before time t₁ is eliminated in a set ratio after disinfection measures are taken at time t₁, Pᵢ(t₁−Δt) is the person safety level of person i at time t₁−Δt, γᵢ(t₁−Δt) is the transmission probability of a infected person to the environment at time t₁−Δt, e^{-τ(t}^{₂}^{t}^{₁}⁾ is the dissipation factor of the source of infection, τ is the attenuation coefficient, and t₂-t₁ is the residence time of the source of infection.

7. The method of claim 2, wherein the distance calculation method between two smart mobile terminals may be given by
$$J ( {RSSI}_{1} , {RSSI}_{2} ) = {10}^{\frac{\vert {RSSI}_{1} + {RSSI}_{2} \vert / 2 - {A}_{1}}{10 {A}_{tt}}} + δ ,$$
where J is the distance between two devices, RSSI₁ is the signal strength from the Bluetooth port of the second mobile terminal to the first one, RSSI₂ is the signal strength from the Bluetooth port of the first mobile terminal to the second one, A₁ is the Bluetooth channel attenuation coefficent under the standard 1 meter distance, Aₜₜ is environmental attenuation factor, δ is environmental correction parameters.

8. The method of claim 2, wherein the infection transmission probability is negatively related to the relative position of the infected person's smart mobile terminal and each smart mobile terminal, and the infection transmission probability is related to the protective measures of the infected person.

9. A system for locating the source of infection, comprising a server and at least one smart mobile terminal;
wherein the server is used to obtain and update a map library, wherein the map library stores the regional risk levels of each location at each time;
the server is used to obtain and update a person database, wherein the person database stores the person safety level of each person at each time;
the smart mobile terminal is used to obtain basic person information and person safety level, and update the person safety level;
the smart mobile terminal is used to display a map interface in real time, wherein the application uses set various colors to mark the regional risk level of each location;
the smart mobile terminal is used to query the historical track of person and person contact history information; and
the smart mobile terminal is used to display the person safety level obtained within the set distance of each smart mobile terminal.

10. A storage medium is characterized in that a computer program is stored in the storage medium, and when the computer program is executed by a processor, each step in the method for locating the source of infection based on big data according to claim 1 is realized.
